(19) United States
(12) Patent Application Publication
(10) Pub. No.: US 2022/0337963 A1
(43) Pub. Date: Oct. 20, 2022

(54) Coupler Device for Round Window Stimulation of the Cochlea

(71) Applicants: Massachusetts Eye and Ear Infirmary, Boston, MA (US); Universität Basel, Petersgraben (CH)

(72) Inventors: Hideko Heidi Nakajima, Andover, MA (US); Christof Theodor Stieger, Liebefeld (CH); Darcy Lynn Frear, Cambridge, MA (US); Hannes Maier, Hannover (DE)

(21) Appl. No.: 17/849,272

(22) Filed: Jun. 24, 2022

Related U.S. Application Data
(63) Continuation of application No. 16/478,917, filed on Jul. 18, 2019.
(60) Provisional application No. 62/448,680, filed on Jan. 20, 2017.

(51) Int. Cl. H04R 25/00; A61N 1/36

(52) U.S. Cl. CPC H04R 25/606; A61N 1/36038; H04R 25/60

(57) ABSTRACT

To account for the anatomical variability of the round window, and its surrounding bony structure, and ensure the safety of the delicate round window membrane (RWM) and structures in the cochlea closely adjacent to the RWM, the disclosure provides devices and methods that safely and effectively couple the motion of a variety of actuators to the RWM.

### CROSS REFERENCE TO RELATED APPLICATIONS

This application is a continuation of U.S. patent application Ser. No. 16/478,917, filed Jul. 18, 2019, which is a 371 U.S. National Phase Application of PCT/US2018/014423, filed on Jan. 19, 2018, which claims priority from U.S. Provisional Application No. 62/448,680, filed on Jan. 20, 2017. The entire contents of the foregoing are incorporated herein by reference.

### GOVERNMENT RIGHTS

This invention was made with Government support under Grant No. DC013303 awarded by the National Institutes of Health. The Government has certain rights in the invention.

### BACKGROUND

Many patients suffer conductive and mixed hearing losses that cannot be treated by conventional hearing aids and surgery alone. One method that has been shown to succeed in treating such patients is by round window (RW) stimulation where transmission of sound to the cochlea is provided by an actuator directly vibrating the RW. Although a number of patients have successfully been treated with RW stimulation after multiple failing surgical interventions as well as various hearing aids, RW stimulation by methods available today has considerable failure due to instability of the prosthetic device and inefficient transmission of sound to the cochlea. A major problem is that these devices used for RW stimulation were not developed for this purpose, but for middle-ear ossicular stimulation, and thus provide inconsistent and inefficient means to transmit sound to the cochlea via the RW. Other proposed methods for actuating RW vibration have risks of traumatizing the RW membrane and sensitive intra-cochlear structures.

### SUMMARY OF THE INVENTION

The present disclosure provides round window (RW) coupler devices, which take into consideration several unique requirements for RW stimulation. A flexible membrane of the RW coupler devices conforms specifically to the anatomy of the RW region of the cochlea, and because the interfacing surface of the coupler is in the form of this soft, flexible membrane, it is much safer and more efficient than prior actuators in transmitting sound to the delicate RW membrane. The coupler devices can be used with existing available actuators.

In one aspect, the disclosure provides coupler devices for transmitting vibration energy to a RW membrane of the cochlea in a mammalian ear, including a hollow housing having a first end and a second end, wherein the hollow housing is dimensioned and configured to fit into a mammalian, e.g., human, middle ear; a first flexible membrane sealed to an opening into the hollow housing adjacent the first end and arranged for contact with an actuator that transmits vibration energy; and a liquid or gel material filling or to be filled into the interior of the hollow housing within the interior of the hollow housing and contacting the flexible membrane. The coupler devices can be manufactured pre-filled with the liquid or gel material, or can be manufactured without the liquid or gel material, which is then filled into the hollow housing either before or after the coupler device is implanted into a patient's middle ear cavity.

In some embodiments, the liquid or gel fills the interior of the hollow housing with minimal or no air space or bubbles. The hollow housing is advantageously dimensioned to be larger in diameter than a patient's RW membrane to avoid trauma to the RW membrane. In some embodiments, the coupler device can further include a soft rubbery material, e.g., a seal, which can be attached or fixed to a perimeter of an opening into the hollow housing at the second end and arranged to contact and seal against surfaces of bone surrounding the RW membrane when the coupler device is implanted.

In certain embodiments, the hollow housing can further include a second flexible membrane sealed against an opening at the second end of the hollow housing for transmitting vibrations to the RW membrane via the liquid or gel material when the coupler device is implanted into a mammalian middle ear cavity at the RW niche.

In some embodiments, the hollow housing is filled with a solid gel material, and the second end of the hollow housing is open to allow the gel material to contact the RW membrane directly when the coupler device is implanted into a mammalian middle ear cavity at the RW niche.

In certain embodiments, the first opening into the hollow housing is an open end at the first end of the hollow housing. In other embodiments, the first opening into the hollow housing is a window opening in a wall of the hollow housing, and wherein the window is positioned and arranged such that an axis perpendicular to the window is at an angle, e.g., a 45, 60, 90, or 120 degree angle, to a central axis of the hollow housing. In such embodiments, the end of the hollow housing is typically sealed with a solid wall of rigid material, e.g., the same material used for the walls of the hollow housing.

In various embodiments, the hollow housing can be tubular and have a cross-section that is a regular geometric shape, e.g., rectangular, triangular, square, pentagonal, or hexagonal, or an irregular shape. In various embodiments, the hollow housing walls can be parallel or non-parallel. The hollow housing can be made of a straight or a bent tubular design to best accommodate the anatomy of a specific patient's middle ear cavity.

In some embodiments that include a second flexible membrane, this second membrane can be configured to balloon out toward and conform to the surrounding area of the RW membrane and RW niche to efficiently couple volume velocity of the fluid in the hollow housing to the RW membrane.

In another aspect, the disclosure includes methods of coupling an actuator force to a RW of the cochlea of a specific patient or subject, e.g., any mammal, including humans, and domesticated animals, as well as other animals. The methods include obtaining any of the coupler devices as described herein; optionally inserting a filler material against the RW membrane to fill the RW niche; implanting the coupler device into the middle ear cavity of the subject so that the second end of the hollow housing is adjacent to the bone surrounding the RW niche and contacts the filler material; mechanically fixing the coupler device within the middle ear cavity to prevent movement of the hollow housing of the coupler within the middle ear cavity; and contacting an actuator to the first flexible membrane sealed to an opening into the hollow housing adjacent to the first end.

In these methods, further steps can include transmitting vibrations with the actuator to cause the liquid or gel material within the hollow housing to transmit the vibrations to the round window membrane.

In some embodiments, the coupler device includes a second flexible membrane sealed against an opening at the second end of the hollow housing for transmitting vibrations to the round window membrane. In other embodiments, the coupler device does not include a second flexible membrane, but instead is filled with a gel material that is sufficiently stable to remain inside the hollow housing.

In certain embodiments, the methods further include sealing a soft rubbery material or seal that is fixed to a perimeter of an opening into the hollow housing at the second end against surfaces of bone surrounding the RW membrane.

In various embodiments, the hollow housing is either filled with a liquid or gel material within the interior of the hollow housing and contacting the flexible membrane before the coupler device is implanted, or the hollow housing is not filled with a liquid or gel material when the coupler device is implanted. In the latter cases, the method further includes, after implanting the couple device, filling the interior of the hollow housing with an amount of a liquid or gel material sufficient for the liquid or gel material to contact the first flexible membrane and, if present, to also contact the second flexible membrane.

The new RW coupler devices and methods provide numerous benefits and advantages. In particular, the new coupler devices improve the transmission of sound via the RW to the cochlea by providing an acoustic coupler that has an adaptable/moldable interface to the RW membrane to prevent loss of volume velocity entering the cochlea. The new coupler devices are also designed to adapt to the geometry of the RW niche to enable efficient transmission of actuator vibration to the RW membrane with minimum of volume velocity leakage. The new coupler devices and methods also enable the use of various actuators that provide vibrations in different directions to be transmitted to the RW membrane, thereby providing improved stability to mount the actuators as best suited for the particular design of actuator. Furthermore, the new coupler devices also improve the stability of the whole RW-stimulation mechanism (including coupler and actuator) to provide a solid and robust interface between the RW membrane and the coupler while maintaining controlled stable positioning. The new coupler devices also mitigate the possibility of damaging the RW membrane by interfacing via a soft moldable structure in the form of a flexible membrane, rather than a stiff mechanical surface found on typical actuators.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF FIGURE DESCRIPTION

FIG. 1 is a schematic diagram of one example of a closed tubular or other shaped (either cylindrical or non-cylindrical) hollow coupler device with parallel walls and flexible membranes, as described herein.

FIG. 2 is a schematic diagram of another example of a tubular or other shaped hollow coupler device with parallel walls, but no flexible membrane adjacent the RW, as described herein.

FIG. 3 is a schematic diagram of another example of a coupler device having a piston-like element to actuate the coupler device.

FIG. 4 is a schematic diagram of another example of a coupler device having non-parallel walls.

FIG. 5 is a schematic diagram of an example of a coupler device design that changes the direction of movement of an actuator compared to the axial motion of the RW membrane.

FIG. 6 is a schematic diagram of a gel-filled coupler device that changes the direction of movement of an actuator as in FIG. 5, but without a flexible membrane adjacent to the RW membrane.

FIG. 7 is a schematic diagram of an off-axis coupler device design having a bent tubular form.

FIG. 8 is a schematic diagram of a coupler device as described herein implanted and mechanically fixed in place at the RW niche in the middle ear cavity, wherein the actuator is arranged to move orthogonally or at some other angle compared to the motion of the RW membrane.

FIG. 9 is schematic diagram of another embodiment of the coupler device as implanted at the RW niche in the middle ear cavity with fascia or other material between a flexible membrane and the RW membrane, wherein the actuator is arranged to move in line with the motion of the RW membrane.

FIG. 10 is a schematic diagram of another embodiment of a coupler device as described herein that includes a piezoelectric disc incorporated into the coupler device.

FIG. 11 is a pair of related graphs that show the results of velocity testing of a coupler device implanted into a middle ear cavity adjacent to the RW niche as described herein.

FIG. 12 is a pair of related graphs that show the results of velocity testing of a commercially available Floating Mass Transducer (FMT) designed for middle ear actuation, but tested herein for direct RW membrane stimulation.

FIG. 13 is a graph that shows a relationship of linearity as demonstrated by plotting stapes velocity versus actuator input voltage at various frequencies for a coupler device as described herein.

FIG. 14 is a graph that shows a relationship of linearity as demonstrated by plotting stapes velocity versus actuator input voltage at various frequencies for an FMT device used for direct RW membrane stimulation.

FIG. 15 is a pair of related graphs that show test results of a comparison of a coupler device as described herein to an FMT device secured in the middle ear cavity with either fascia or dental impression material (Jeltrate™).

Like reference symbols in the various drawings indicate like elements.

### DETAILED DESCRIPTION

The present disclosure provides RW coupler devices that take into consideration several unique requirements for RW stimulation. These requirements include specific dimensions to enable the coupler to stimulate the RW membrane and not the surrounding bone. In addition, the devices include a flexible membrane that stimulates yet avoids trauma to the RW membrane, rather than a small, stiff surface area actuator (smaller than the diameter of the RW membrane) directly interfacing the RW membrane, which can traumatize the RW membrane. Furthermore, the devices enable the use of actuators that vibrate in an orthogonal or other angle with respect to the RW membrane for optimal RW stimulation. In addition, the new coupler devices avoid loss of volume velocity by use of a relatively large surface area flexible membrane interface rather than a small surface area actuator interface.

Another advantage is that the new coupler devices do not require significant tension on the RW membrane, as has been shown necessary with other actuators. The coupler device diameter that interfaces the RW membrane is also larger than the bony rim perimeter of the RW membrane, preventing inadvertent trauma and tension to the RW membrane. The coupler devices described herein also avoid the need for excessive drilling of the bone surrounding the RW membrane, which can result in trauma to the RW membrane. In addition, the coupler devices can be used with a wide variety of available actuators.

The RW coupler devices serve as a hydraulic/acoustic interposition element that is implanted between a transducer/actuator and a middle/inner ear structure. The coupler devices include an acoustically rigid and biocompatible hollow housing (made of materials such as titanium, stainless steel, brass, ceramic, or other rigid biologically inert materials, such as rigid plastics, e.g., acrylics, nylons, and polyetheretherketones (PEEK), approved for medical implantation) with two open ends. The tubular housing can have a cross-section that is circular, square, rectangular, or other shape, and the two open ends can have the same or different sizes. The coupler devices also include at least two mobile coupling surfaces in the form of flexible membranes that are sealed against the open ends of the tubular or other shaped housing. A first flexible membrane provides an actuator input surface and the second flexible membrane provides an output surface that couples vibrations from the actuator to the inner ear via the RW.

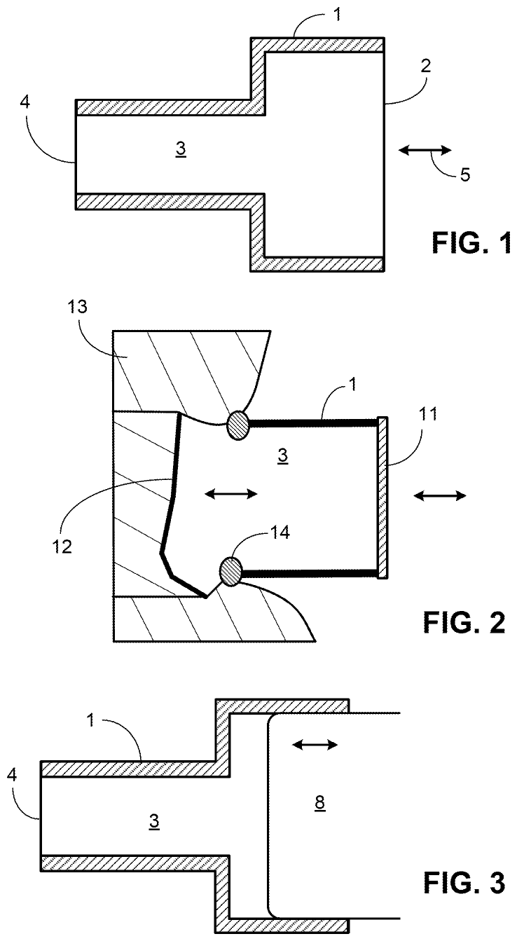

In one configuration as shown in FIG. 1, the coupler device includes a housing (1) that encloses a tubular or other non-cylindrical-shaped lumen. The housing can have smaller, larger, same, or different diameters at the open ends to adapt one open side (2) to the actuator (5) and on the other open side (4) to the stimulated ear structure, which is typically the RW membrane. Depending on the type of actuator used, the cross-sectional area of the opening (2) adjacent the actuator (5) can be larger than the interfacing surface (4) to reduce the stroke (distance) that the actuator would have to move yet allow for large volume velocity driving the RW membrane.

The tubular or other shaped housing can be hermetically sealed by flexible membranes on each side (2, 4) and filled with a biocompatible fluid or gel (3, 10) of low compressibility and of appropriate acoustic properties such as low shore biocompatible silicone or gel (e.g., thiolene gel that is composed of diacrylates and polythiols, which are mixed to create a liquid that gels over time allowing for specified material properties such as stiffness) to allow the least loss of volume velocity from (2) to (4). The flexible membranes can be made of, for example, silicone used in medical application, e.g., polydimethylsiloxanes (PDMS)-based materials. Any other flexible biocompatible materials can be used. For example, on the actuator side of the coupler device, stiffer membranes can be used that can be made, for example of very thin metal disks, e.g., of titanium.

If a gel is used, a non-hermetically sealed rigid lumen is a possible alternative if the gel is sufficiently stiff and stable so as not to leak out of the device, and if the gel adheres to the edges of the coupler. In such embodiments, the coupler device can consist entirely of a gel-filled housing, where the housing is designed to fill the RW niche space once implanted and the gel then contacts the RW membrane (12) directly as shown in FIG. 2. Additionally, to seal and conform to the unevenness of the RW niche (bone surrounding he RW membrane), a rubbery conforming substance (14) can be attached to the rim of the housing that abuts the bone (13) surrounding the RW membrane. For the hollow coupler housing, a 3D-printed housing designed specifically for a given patient's middle ear anatomy can be made. In some embodiments, the housing of the device can be made in advance and the gel is injected once the device is implanted into the middle ear.

In all embodiments, the actuator side (2) of the coupler device is closely attached to an actuator, such as a commercially available transducer such as Floating Mass Transducer (FMT) device, Middle Ear Transducer (MET) device, or Direct Acoustic Cochlear Stimulation (DACS) device (designed for middle ear actuation, though some have been used or proposed for RW stimulation), or other actuators (5) that provide vibration to the coupler. Alternatively, the actuator (5) is attached directly or by a solid plate (11) to increase the effective vibrating surface area to the transmitting material.

As shown in FIG. 2, the interface between actuator (5) and coupler can also be designed as a piston-like system in which a rigid thin plate or disk (e.g., of thin plastic or metal) (11) is secured to the flexible membrane at this end of the housing as a contact point for the actuator (5) or an extension of the actuator, and moves in a piston-like motion. In these embodiments, the actuator can directly stimulate the plate or disk on the flexible membrane, which, in turn, stimulates the liquid or gel in the housing. Alternatively, the piston-like element (8) can directly contact the RW membrane (12), if the membrane is sufficiently robust for such direct contact. In other embodiments as shown in FIG. 3, the end of a piston-like element (8) is designed to fit sealingly into the opening of the housing to serve as a piston without the need for a flexible membrane. In these embodiments, the end of the piston (8) directly stimulates the liquid or gel within the housing. If liquid fills the housing, the piston (8) would need to be sealed to the coupler's outer housing (1) to prevent liquid leakage and entrance of air into the coupler device. This piston (8) can then be driven by an actuator, or the actuator could be the piston itself.

The whole apparatus (coupler device and actuator) can be principally stabilized, for example, by conventional hardware and/or medical adhesives that are used in otologic surgery, craniofacial, maxillofacial surgery, and/or reconstructive surgery. For example, the coupler device can be stabilized by filling between it and surrounding middle-ear cavity wall with glue, 3-D printed material, etc. The actuator can be positioned and stabilized by a bracing apparatus such as used with known DACS or similar devices using bone-plate hardware.

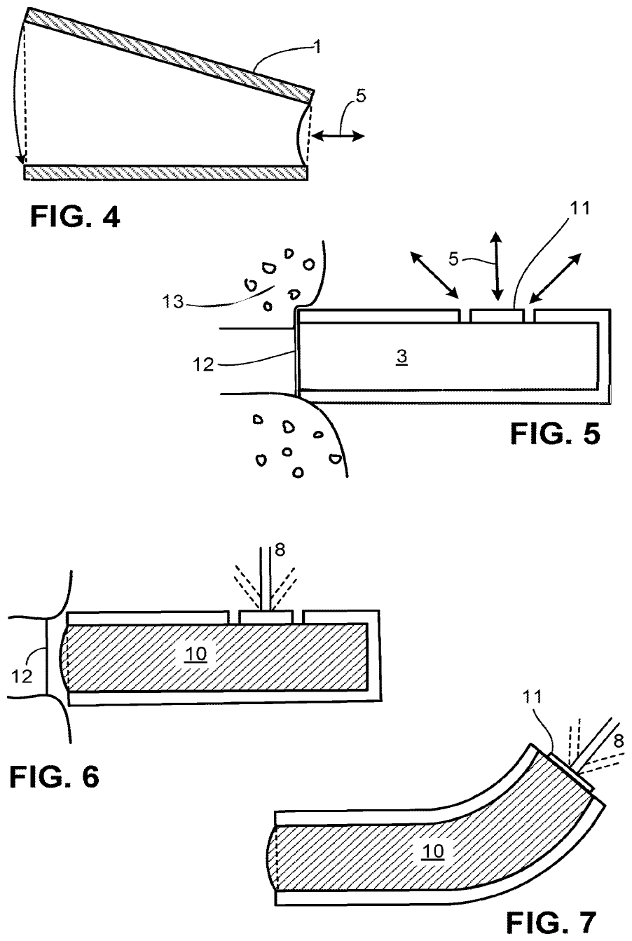

As shown in FIG. 4, other shapes of the rigid housing (1) are possible. The housing can be cylindrical, non-cylindrical, symmetrical, or non-symmetrical in shape. The side that interfaces with the actuator (5) can be larger or smaller than the side that interfaces with the RW membrane.

In another embodiment shown in FIG. 5, the design of the coupler device serves not only to adjust the diameter of the actuator to the stimulated structure (maximizing transmission of volume velocity), but also to change the direction of movement of the actuator as compared to the motion of the RW, minimizing losses due to vector decomposition. This embodiment of the coupler device has a flexible membrane at one open end of a hollow housing that interfaces the RW membrane (12). The hermetically sealed, fluid or gel filled inner volume (3) is actuated by a flexible membrane “window” in the housing that is not in the axial direction (i.e., not in line with a central axis of the direction of the motion of the RW membrane). To increase the effective surface area an optional hard plate (11) can be positioned on the flexible window that is driven by an actuator.

In the embodiment shown in FIG. 5, the actuator interface is arranged orthogonal to the RW interface, which should make it easier to mount the actuator in a direction for stimulation that is more feasible or accessible for the actuator due to anatomical constraints. Also, the other end of the coupler (opposite to the side interfacing the RW) can then be used to attach to hardware stabilizing the coupler to the surrounding wall of the middle ear cavity.

In any of these embodiments, the actuator (5) may optionally not be fixed to the flexible window or plate, to allow vibrational input adjustment to accommodate the available angular range of the anatomical constraints and actuator shape and design (e.g. allowing vibration in the off-axis direction). This is illustrated in FIG. 5 by the three separate arrows at different angles.

In a similar embodiment shown in FIG. 6, the coupler device requires no outer hermetic seal at the end facing the RW membrane (as in the embodiment of FIG. 2), and the transmission of vibrations is performed by a silicone element or gel (10) that is mechanically stable in an acoustically rigid housing analogous to the embodiments described above. In these embodiments, the stable gel adheres to the inner walls of the housing and also to the outer edge of the opening adjacent to the RW membrane. The gel can thus directly interface the RW membrane. If the gel is not sufficiently stable and is thus too fluid, a thin flexible membrane can also be used as in the other embodiments. As in FIG. 5, the embodiment of FIG. 6 can include a rigid disk or hard plate (11) that is contacted by an actuator (5), which can move at various angles with respect to the disk or plate (shown by dashed lines in FIG. 6).

Alternatively, an off-axis actuator input design can be implemented as a bent tubular or other shaped hollow housing as shown in FIG. 7, instead of a straight tubular or other shaped housing. FIG. 7 illustrates this approach as a silicone (10) filled design without a flexible membrane, but can be of the hermetically sealed membrane design with flexible membranes at both ends as described as shown in the embodiments of FIGS. 1 and 3 to 5.

The embodiments illustrated in FIGS. 5 to 7 are shown with approximate equally sized acoustical-mechanical input and output areas at the ends or sides of the hollow housings, but these ends or side “windows” can also have different sizes for hydraulic amplification and/or as impedance transformers.

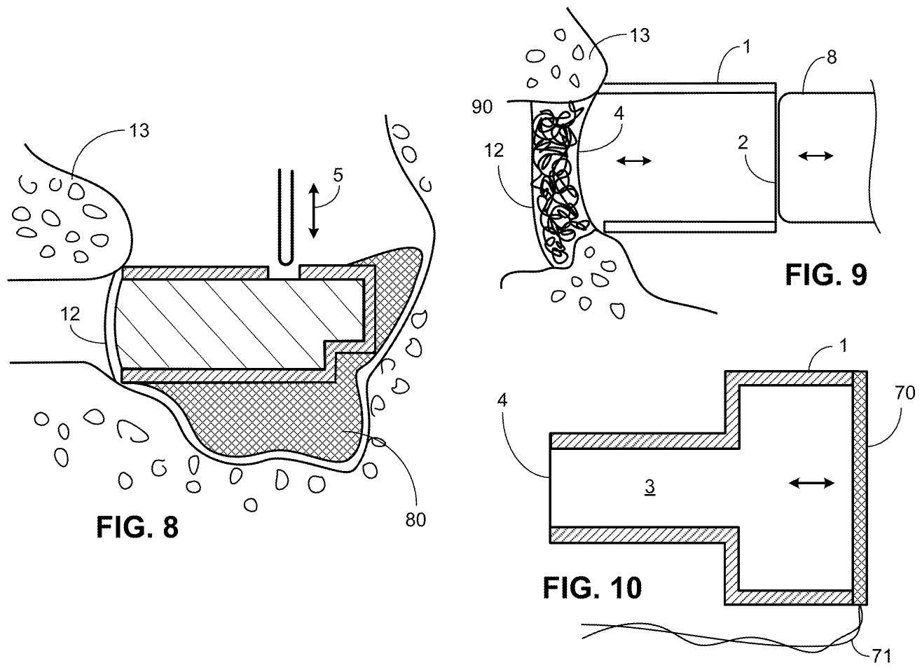

In use, the coupler devices will be mechanically fixed in place to be stable to allow a reliable interface to the RW membrane (12) by contacting the flexible membrane or solid gel against the bone (13) surrounding the RW niche as shown in FIG. 8. This implantation and sealing of the flexible membrane or solid gel against the bone can be performed by stabilizing the coupler device by conventional methods often used for middle-ear active prostheses, such as metal mounting hardware. It is also possible to use various standardized collections of molds, or use standard surgical glues such as cement, fibrin glue, epoxy, bone pate, and other materials (80) used in otologic and neurological surgeries to secure the hollow housing in the precisely desired location within the middle ear.

Alternatively, a custom mold to surround the coupler device can be made (with 3D printed material shaped based on imaging studies beforehand) and then the coupler device and custom molded material is implanted into the patient's middle ear using standard surgical techniques.

FIG. 9 is a schematic diagram of another embodiment of the coupler device as implanted at the RW niche in the middle ear cavity with fascia or other material (90) between a flexible membrane (4) and the RW membrane (12), wherein the actuator is arranged to move in line with the motion of the RW membrane.

Other modifications include the use of customized actuators that are incorporated as part of the coupler device and then connected to a source of vibrations once implanted into the middle ear. For example, as shown in FIG. 10, in some embodiments a piezoelectric disc is incorporated into the coupler device. The piezoelectric disc would be mounted to the device (70) and connected via wires (71) to a voltage source. The disc is arranged to move with a stroke to elicit the same force as an external actuator. The coupler device would be stabilized in a similar manner as the other coupler devices described herein.

### EXAMPLES

The invention is further described in the following examples, which do not limit the scope of the invention described in the claims.

### Example 1—Testing of Coupler Device in Human Cadaveric Temporal Bones

Prototype moldable coupler devices for RW stimulation were tested in fresh cadaveric human temporal bones, which include the entire ear (outer, middle, and inner ear portions), and thus provide the same general mechanical properties of the ear as in living humans. The resulting measurements show efficient transmission of sound to the cochlea over a wide frequency bandwidth, with a large dynamic range and good linearity, while preventing the risk of trauma to the delicate RW membrane.

We directly compared the performance of an embodiment of the coupler devices described herein to a current RW stimulation device (Floating Mass Transducer—FMT by Med El). In the same ear, sound transmission of the implanted coupler device results in significantly larger efficiency of sound transmission, larger bandwidth, and wider dynamic range when compared to direct stimulation of the RW with an actuator of an FMT device. The FMT device also caused large distortions (non-linearity) of sound transmission while the coupler device described herein provided excellent fidelity with linearity. In addition, the coupler devices described herein are mechanically more stable and much safer for the RW membrane and intra-cochlear structures than the actuator of an FMT device, and should provide an effective way to stimulate the RW membrane and to enable transmission of sound to the cochlea even in patients with refractory conductive and mixed hearing loss.

Fresh human cadaveric specimens are extracted (donated with permission specifically for research) within 24 hours post mortem, then immediately frozen or used within two days (Nadol, J. B. & McKenna, M. J., “Surgery of the Ear and Temporal Bone.” (Lippincott Williams & Wilkins, 2005). These specimens have similar macro-mechanical properties to the living tissue and are prepared in a manner described in previous publications (Stieger et al., “Comparison of forward (ear-canal) and reverse (round-window) sound stimulation of the cochlea,” Hear. Res., 301, 105-114 (2013); Nakajima et al., “Evaluation of round window stimulation using the floating mass transducer by intracochlear sound pressure measurements in human temporal bones,” Otol. Neurotol. Off. Publ. Am. Otol. Soc. Am. Neurotol. Soc. Eur. Acad. Otol. Neurotol., 31, 506-511 (2010); and Nakajima et al., “Performance considerations of prosthetic actuators for round-window stimulation,” Hear. Res., 263, 114-119 (2010)). Posterior tympanotomy with entrance to the middle-ear cavity via facial recess is drilled to provide access to the stapes and RW area. AC response of stapes and RW velocities (Vstaₚ and VRw) are measured with laser Doppler vibrometry (Polytec CLU 1000) to check for half cycle phase relationship between the velocities to ensure that air did not enter into the inner ear and that there is no fluid leak (Merchant et al., “Middle ear mechanics of Type III tympanoplasty (stapes columella): II. Clinical studies,” Otol. Neurotol. Off. Publ. Am. Otol. Soc. Am. Neurotol. Soc. Eur. Acad. Otol. Neurotol., 24, 186-194 (2003)). After confirming good integrity of the specimen, RW stimulation is performed with a stimulus voltage (either iso-intensity voltage or varying voltages to provide iso-vibrational motion) between 0.2-10 kHz presented to a piezoelectric stack actuator or FMT.

The coupler device was made of a brass tubular housing about 2 mm in length and 2.5 mm in diameter (with a rigid wall width of 0.225 mm). The thin flexible membranes attached to each of the two sides were made with a cured photopolymer (Norland optical adhesive 68, with ultraviolet curing, which is a urethane-based formulation of tetrahydrofurfuryl and mercapto-ester). The coupler device was filled with water ensuring no air bubbles. The coupler device was placed at the opening of the RW niche with fascia between the coupler and RW membrane to fill the volume of the niche as shown in FIG. 10. The fascia enabled better transmission volume velocity from the coupler device to the RW membrane.

The flexible membrane that interfaces the RW was ballooned out to mold to its surroundings and to interface with the delicate RW membrane (ballooning out approximately 0.8 mm), requiring some drilling of the bony RW overhang (approximately 3 mm of overhang). The other flat flexible membrane was mechanically stimulated by a rod (about 2 mm in diameter) attached to a stack ceramic piezo-electric actuator.

The FMT was implanted into the same middle ear cadaveric model, using standard surgical techniques in an ideal manner to ensure good RW coupling and stabilization of the FMT. The FMT actuator rested against the bony surface surrounding the RW niche. However, depending on the size of the RW, the FMT can be larger in diameter than the RW diameter, requiring drilling of the RW niche. Drilling could cause the RW membrane to lift off at the edges, because there is continuation of RW membrane tissue on the surface of the bony overhang. To hold the FMT in place, the device is wrapped in fascia. However, some reports indicate this does not always properly secure the device.

Comparisons were made between the FMT and the RW coupler device by testing the two RW stimulation methods on the same cadaveric human ear. The Vₛₜₐₚ was recorded in response to RW stimulation using laser Doppler vibrometry. The V_{act} was also recorded with a laser Doppler vibrometer. Vₛₜₐₚ driven by RW stimulation is used to measure transmission of sound from the stimulator through the cochlear fluid to the stapes. The velocity ratio between Vₛₜₐₚ and FMT (V_{FMT}), and the velocity ratio between Vₛₜₐₚ and actuator (V_{act}) with the coupler device were compared. The velocity of the cochlear promontory was measured to determine any vibration induced by the stimulator. The velocity of the cochlear promontory was measured to determine any vibration induced by the stimulator to ensure that we were not stimulating the entire bony otic capsule (as in bone conduction). We wanted to ensure that only the fluid and flexible membrane are significantly vibrating.

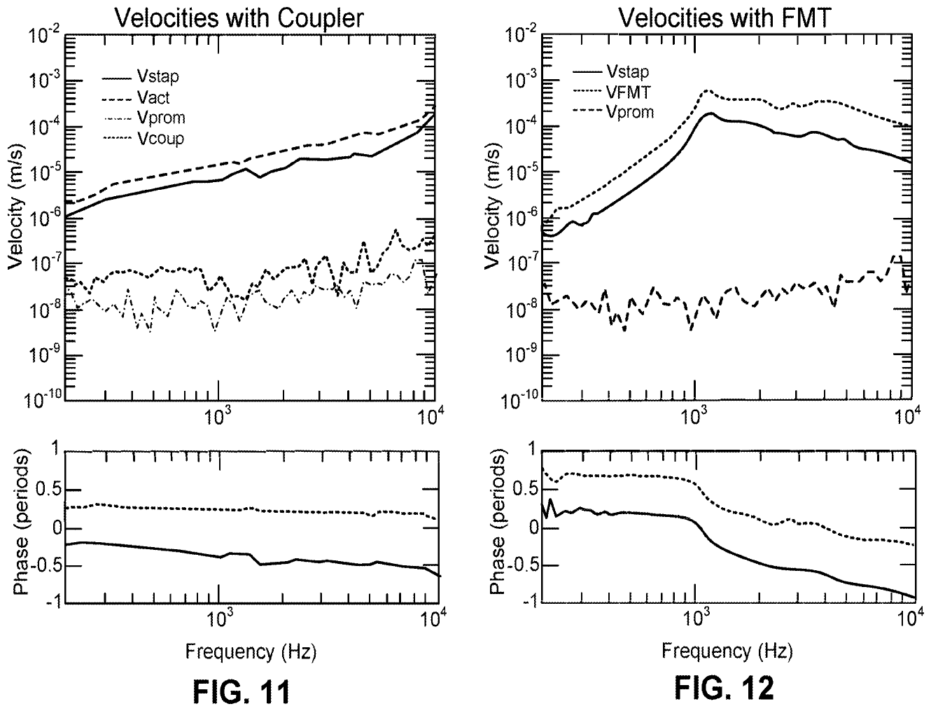

FIGS. 11 and 12 show the velocity responses to iso-intensity voltage stimulation between 0.2-10 kHz to the piezoelectric stack actuator (left, 0.07 Vrms) and to the FMT actuator (right, 0.03 Vrms). The piezoelectric actuator used with the coupler device increased in velocity with frequency, while the FMT device velocity peaked around 1.2 kHz. Stapes, actuator, and FMT velocities were well above the motion of the bony promontory. The coupler cylinder motion was significantly lower than the stapes and actuator velocities, similar to the bony promontory. The stapes velocity magnitudes generally followed the actuator or FMT velocities. The phases of the stapes velocity and actuator motion were generally different by a half cycle.

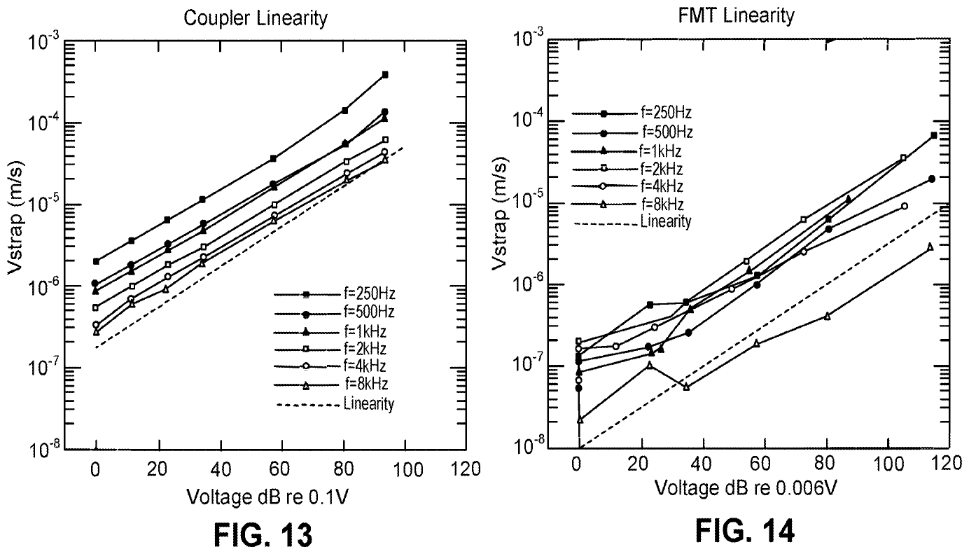

FIGS. 13 and 14 show the relationship of linearity as demonstrated by plotting stapes velocity versus the actuator input voltage at various frequencies (as a reference, a linear relationship is shown with a dashed line) for the coupler and FMT device, respectively. Thus, FIG. 13 shows that the coupler device functioned linearly for a wide bandwidth and dynamic range. On the other hand, FIG. 14 shows that the FMT device was near linear for only a limited dynamic range and frequency range (near 2 kHz). The FMT was not linear at low and high frequencies. From these results, it is clear that the coupler device can be used with a variety of actuators, and if that actuator is linear, the coupler device will not distort the mechanical motion for a large dynamic range and bandwidth.

The volume velocity ratio, i.e., the ratio of the stapes volume velocity and the actuator volume velocity (Uₛₜₐₚ/U_{act}), was estimated by:

$$\frac{{V}_{stap} * {A}_{stap}}{{V}_{act} * {A}_{act}} ,$$

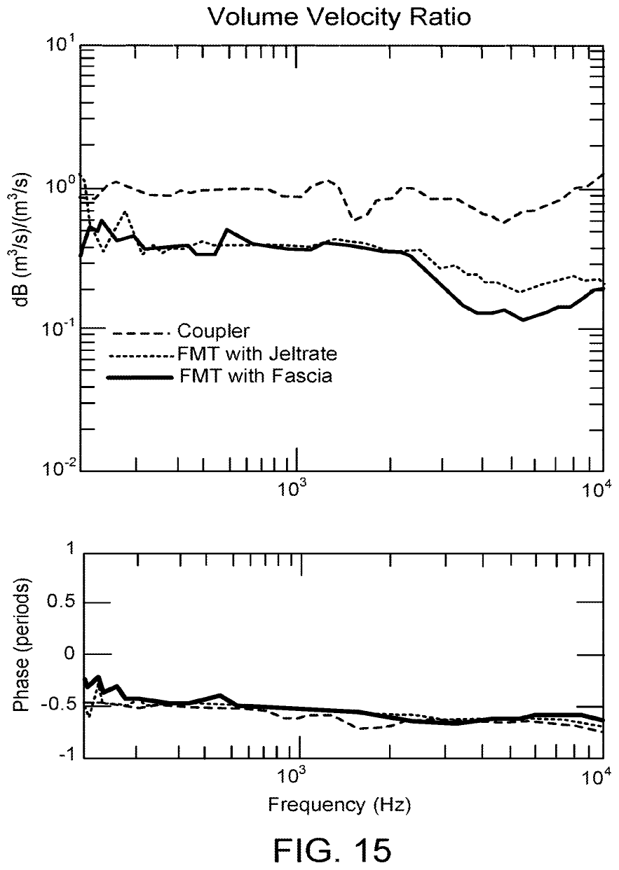

and
the ratio of the stapes volume velocity and FMT volume velocity (Uₛₜₐₚ/U_{FMT}) was estimated by:

$$\frac{{V}_{stap} * {A}_{stap}}{{V}_{FMT} * {A}_{FMT}}$$

wherein Aₛₜₐₚ=3.2 mm², A_{act}=1.8 mm², and A_{FMT}=2.5 mm². FIG. 15 is a graph that shows the testing, which indicated that the volume velocity ratio was higher with the coupler than the FMT device. In particular, FIG. 15 shows the volume velocity ratio for the coupler device vs. the FMT with Jeltrate™ brand dental impression material that is a substitute for fascia and vs. the FMT with natural fascia in dB. The dental impression material cures to a rubbery and stiffer consistency than fascia. With the FMT positioned ideally at the RW niche and braced in a stable manner, the dental impression material performed better than the softer fascia above 2 kHz (see FIG. 13).

The coupler device provides effective transfer of volume velocity (Uₛₜₐₚ/U_{act}). Results showed that RW stimulation with the coupler device as described herein has a higher volume velocity ratio than the FMT device. Furthermore, stimulation with the coupler device provides linear results for a large dynamic range and wide frequency bandwidth. On the other hand, the FMT device exhibited distortion at most frequencies, limiting the dynamic range and bandwidth of its performance.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A coupler device for transmitting vibration energy to a round window membrane of the cochlea in a mammalian ear, comprising
an acoustically rigid and biocompatible hollow housing having a first end and a second end, wherein the hollow housing is dimensioned and configured to fit into a mammalian middle ear and wherein the second end has a diameter larger than a bony rim perimeter of the round window membrane in the mammalian ear;
a piston-like element designed to fit sealingly into a first opening into the hollow housing at the first end and arranged for contact with an actuator that transmits vibration energy;
a flexible membrane sealed to a second opening into the hollow housing at the second end and arranged to transmit vibrations to the round window membrane when the coupler device is implanted; and
a liquid or gel material filling or to be filled into the interior of the hollow housing between the flexible membrane and the piston-like element.

2. The coupler device of claim 1, wherein the liquid or gel fills the interior of the hollow housing with minimal air space or bubbles.

3. The coupler device of claim 1, wherein the device further comprises a soft rubbery material fixed to a perimeter of the second opening into the hollow housing at the second end and arranged to contact and seal against surfaces of bone surrounding the round window membrane.

4. The coupler device of claim 1, wherein the hollow housing is filled with the liquid material.

5. The coupler device of claim 1, wherein the hollow housing is filled with the gel material.

6. The coupler device of claim 1, wherein the first opening into the hollow housing is a window opening in a wall of the hollow housing, and wherein the window opening is positioned and arranged such that an axis perpendicular to the window opening is at an angle to a central axis of the hollow housing.

7. The coupler device of claim 6, wherein the axis perpendicular to the window opening is arranged to be perpendicular to the central axis of the hollow housing.

8. The coupler device of claim 1, wherein the hollow housing is tubular.

9. The coupler device of claim 1, wherein the hollow housing cross-section is a regular geometric shape.

10. The coupler device of claim 1, wherein the hollow housing has a configuration of a bent tubular structure.

11. The coupler device of claim 1, wherein the flexible membrane is configured to balloon out toward and conform to the surrounding area of the round window membrane and round window niche to efficiently couple volume velocity of the fluid or gel material in the hollow housing to the round window membrane.

12. A method of coupling an actuator force to a round window of the cochlea, the method comprising
obtaining a coupler device of claim 1;
inserting a filler material against the round window membrane to fill the round window niche;
contacting an actuator to the piston-like element at the first end of the rigid hollow housing;
implanting the coupler device into the middle ear cavity so that the second end of the rigid hollow housing is adjacent to bone surrounding the round window niche such that the flexible membrane can contact the filler material; and
mechanically fixing the coupler device within the middle ear cavity to prevent movement of the hollow housing of the coupler device within the middle ear cavity.

13. The method of claim 12, further comprising transmitting vibrations with the actuator to cause the liquid or gel material within the hollow housing to transmit the vibrations to the round window membrane.

14. The method of claim 12, further comprising sealing a soft rubbery material fixed to a perimeter of the second opening into the hollow housing at the second end against surfaces of bone surrounding the round window membrane.

15. The method of claim 12, wherein the hollow housing is filled with a liquid or gel material within the interior of the hollow housing between the piston-like element and the flexible membrane before the coupler device is implanted.
